(19) United States
(12) Patent Application Publication
(10) Pub. No.: US 2003/0114813 A1
(43) Pub. Date: Jun. 19, 2003

(54) High capacity and high rate absorbent composite

(76) Inventors: Richard Norris Dodge II, Appleton, WI (US); Sridhar Ranganathan, Suwanee, GA (US); Roland Columbus Smith JR., Gainesville, GA (US)

Correspondence Address: KIMBERLY-CLARK WORLDWIDE, INC., 401 NORTH LAKE STREET, NEENAH, WI, 54956

(21) Appl. No.: 10/022,329

(22) Filed: Dec. 14, 2001

(51) Int. Cl. A61F 13/15; A61F 13/20

(52) U.S. Cl. 604/368; 604/372

(57) ABSTRACT

There is provided a new structural composite comprising a web of Z-directionally oriented fibers. The web must contain a large percentage of superabsorbent fibers and the balance of the fibers may be synthetic or natural fibers. Binder fibers are present and should be present in an amount of between 10 and 45 weight percent, more particularly about 30 weight percent. Polyethylene/polypropylene side-by-side or sheath/core bicomponent fibers are particularly well suited for this purpose. Natural fibers or synthetic fibers that are hydrophilic may also be added to the web in order to control the wettability of the web. Pulp, cotton, and Rayon are suitable for this purpose and may be present in an amount between 0 and 40 weight percent, more particularly about 20 to 30 percent. These materials are suitable for use in personal care products like diapers, training pants, incontinence products, bandages, and sanitary napkins.

### BACKGROUND OF THE INVENTION

The present invention concerns formed materials mainly for personal care products like diapers, training pants, swim wear, absorbent underpants, adult incontinence products and feminine hygiene products. This material may also be useful for other applications such as, for example, in bandages and wound dressings, nursing pads and in veterinary and mortuary applications.

One of the problems identified in the field of personal care articles has been the issue of combined high intake rate and high capacity. Intake rate is important because a low intake rate can result in liquid run-off. Run-off can cause staining of the wearer's clothing or bedding. A high capacity is important, particularly for products to be worn overnight, so that large quantities of liquid may be contained. A combination having both of these characteristics at high values has been difficult to achieve. Clearly, a product having a high intake rate but low capacity would need to be discarded and replaced quite regularly, resulting in great expense and effort on the part of the wearer. A product having a high capacity for liquid but a very low intake rate would likewise be unsatisfactory since liquid would not be absorbed by the product and this would result in staining of clothing and/or bedding.

There remains a need in the art for a material for use in personal care products which will intake liquids quickly and also have a very high capacity. This combination of characteristics would improve overall leakage performance of products as well as consumer satisfaction.

### SUMMARY OF THE INVENTION

In response to the discussed difficulties and problems encountered in the prior art, a new structural composite comprising a web of Z-directionally oriented fibers is provided. The web must contain a large percentage of superabsorbent fibers; at least 40 weight percent and at most 90 weight percent, more particularly more than 50 percent and still more particularly more than 60 percent. The balance of the fibers may be synthetic or natural fibers. Synthetic fibers include polymeric fibers like polyolefins, polyamides, polyesters, polyethers, polyethylene terephalate and combinations thereof in bicomponent form.

A binder is used in an effective amount to maintain the integrity of the web. The binder is preferably a bicomponent fiber and should be present in an amount of between 10 and 60 weight percent, more particularly about 20 to 50 weight percent and most preferably about 30 percent by weight. Polyethylene/polypropylene side-by-side or sheath/core bicomponent fibers are particularly well suited for this purpose. Binders in powder and liquid forms may also be used in this invention.

Natural fibers or synthetic fibers that are hydrophilic may also be added to the web in order to control the wettability of the web. Pulp, cotton, and Rayon are suitable for this purpose and may be present in an amount between 0 and 40 weight percent, more particularly about 20 to 30 percent.

Synthetic fibers such as PET may be added to improve the resiliency of the web to provide enhanced intake function. They may be in the range of 10 to 40 weight percent, more particularly 20 to 30 percent, if desired.

Particulate superabsorbents may optionally also be added to the web to provide added capacity while maintaining intake function. Superabsorbents such as FAVOR® SXM 880 available from Stockhausen in Greensboro, N.C., and Drytech® 2035 available from Dow Chemical in Midland, Mich. are two examples.

The nonwoven material of this invention should have an intake rate at 50 percent saturation of at least 7 ccl/s and a capacity of at least 4 g/g, more particularly an intake rate of at least 8 cc/s and a capacity of at least 6 g/g, and still more particularly an intake rate of at least 8 cc/s and a capacity of at least 10 g/g.

Specific embodiments of the invention include; a nonwoven material for personal care products made from superabsorbent fibers in an amount of at least 50 weight percent, polypropylene/polyethylene bicomponent fiber in amount of about 20 weight percent, and natural fibers, in a Z-directionally oriented web; a nonwoven material for personal care products made from superabsorbent fibers in an amount of at least 60 weight percent, polypropylene/polyethylene bicomponent fiber in amount of about 30 weight percent, and rayon fibers, in a Z-directionally oriented web and; a nonwoven material for personal care products made from superabsorbent fibers in an amount of at least 50 weight percent, polypropylene/polyethylene bicomponent fiber in amount of about 20 weight percent, and natural fibers, in a Z-directionally oriented web, where the web has an intake rate at 50 percent saturation of at least 7 cc/s and a capacity of at least 4 g/g.

These materials are suitable for use in personal care products like diapers, training pants, incontinence products, bandages, and sanitary napkins.

### BRIEF DESCRIPTION OF THE FIGURES

FIG. 1 is a diagram of a vibrating lapper used to produce webs having perpendicularly laid (Z-directional) fibers.

FIG. 2 is a diagram of a rotary lapper used to produce webs having perpendicularly laid (Z-directional) fibers.

FIG. 3 is a graph of intake rate versus capacity for a number of samples.

FIG. 4A (side view) and 4B (top view) are drawings of an apparatus for measuring the intake rate and capacity of a web.

FIG. 5 is a drawing of a vacuum apparatus used in testing the saturation of a material.

### DEFINITIONS

As used herein the term “nonwoven fabric or web” means a web having a structure of individual fibers or threads which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

“Spunbonded fibers” refers to small diameter fibers that are formed by extruding molten thermoplastic material as filaments from a plurality of fine capillaries of a spinneret. Such a process is disclosed in, for example, U.S. Pat. No. 4,340,563 to Appel et al. and U.S. Pat. No. 3,802,817 to Matsuki et al. The fibers may also have shapes such as those described, for example, in U.S. Pat. No. 5,277,976 to Hogle et al. which describes fibers with unconventional shapes.

“Bonded carded web” refers to webs that are made from staple fibers which are sent through a combing or carding unit, which separates or breaks apart and aligns the staple fibers in the machine direction to form a generally machine direction-oriented fibrous nonwoven web. This material may be bonded together by methods that include point bonding, through air bonding, ultrasonic bonding, adhesive bonding, etc.

“Perpendicularly laid” or “Z-directional fabrics” are fabrics in which the fibers are oriented in a direction perpendicular to the predominant plane (X-Y) of the fabric. This predominant plane is also generally the MD-CD plane. This refers to fabrics wherein the fibers are predominately oriented in the Z-direction during the formation of the fabric, as opposed to during a post-treatment step like creping. Examples of such materials and methods may be found in PCT publications WO 00/66057 and WO 00/66284, corresponding to U.S. applications Ser. Nos. 09/538,744 and 09/537,564, respectively, and both commonly assigned.

“Hydrophilic” describes fibers or the surfaces of fibers that are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials can be provided by a Cahn SFA-222 Surface Force Analyzer System, or a substantially equivalent system. When measured with this system, fibers having contact angles less than 90° are designated “wettable” or hydrophilic, while fibers having contact angles equal to or greater than to 90° are designated “nonwettable” or hydrophobic.

As used herein, through-air bonding or “TAB” means a process of bonding a nonwoven bicomponent fiber web in which hot air is forced through the web. The temperature of the air is sufficient to melt one of the polymers of which the fibers are made. The air velocity is usually between 100 and 500 feet per minute and the dwell time may be as long as 6 seconds. The melting and resolidification of the polymer provides bonding. Through-air bonding (TAB) requires the melting of at least one component to accomplish bonding, so it is usually restricted to webs with two components like conjugate fibers or those which include an adhesive. In the through-air bonder, air having a temperature above the melting temperature of one component and below the melting temperature of another component is directed from a surrounding hood, through the web, and into a perforated drum supporting the web.

Alternatively, the through-air bonder may be a flat arrangement wherein the air is directed at an angle through the web. The operating conditions of the two configurations are similar, the primary difference being the geometry of the web during bonding. The hot air melts the lower melting polymer component and thereby forms bonds between the filaments to integrate the web. “Personal care product” means diapers, training pants, swim wear, absorbent underpants, adult incontinence products, bandages and feminine hygiene products. It may further encompass veterinary and mortuary products.

### TEST METHODS AND MATERIALS

Basis Weight: A circular sample of 3 inches (7.6 cm) diameter is cut and weighed using a balance. If a 3 inch diameter sample cannot be obtained one of smaller diameter may be used. Weight is recorded in grams. The weight is divided by the sample area. Five samples are measured and averaged.

Material caliper (thickness): The caliper of a material is a measure of thickness and is measured at 0.05 psi (3.5 g/cm²) with a STARRET® bulk tester, in units of millimeters. Samples are cut into 4 inch by 4 inch (10.2 cm by 10.2 cm) squares and five samples are tested and the results averaged.

Density: The density of the materials is calculated by dividing the weight per unit area of a sample in grams per square meter (gsm) by the material caliper in millimeters (mm). The caliper should be measured at 0.05 psi (3.5 g/cm²) as mentioned above. The result is multiplied by 0.001 to convert the value to grams per cubic centimeter (g/cc). A total of five samples would be evaluated and averaged for the density values.

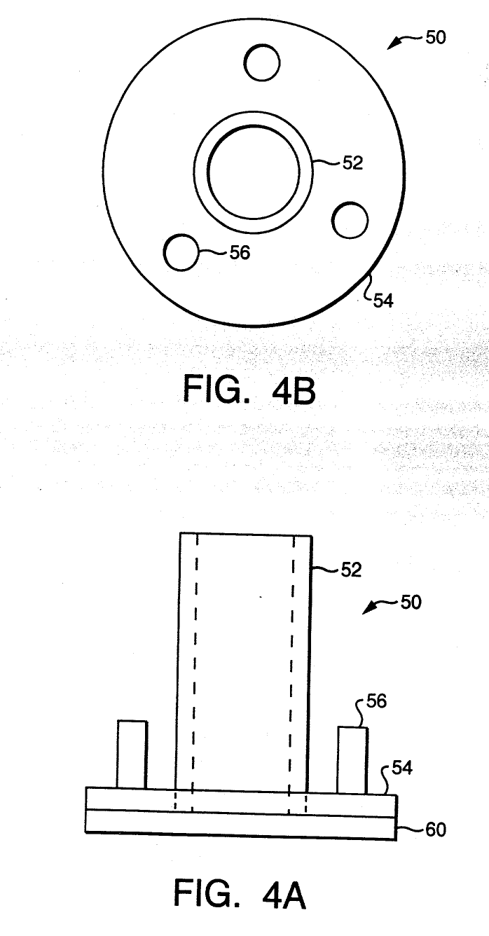

Intake Rate and Capacity: Intake rate is determined by weighing a 3 inch (7.6 cm) diameter sample of absorbent composite, placing the sample 60 in an apparatus and applying 250 g of weight uniformly around the cylinder of the apparatus to apply pressure to the sample 60. The apparatus 50 is shown in FIGS. 4A (side view) and 4B (top view) and has a LEXAN® plate 54 with a cylinder 52 attached to it in its center, and weights 56 arranged around the cylinder 52. The plate 54 is 76 mm (3 inches) in diameter and 5.56 mm (7/32 inch) thick and has a 25.4 mm (1 inch) hole in its center. The cylinder 52 has an inside diameter of 25.4 mm, a wall thickness of 3.18 mm (⅛ inch) and a height of 69.9 mm (2.75 inch). The apparatus 50 has a mass of 39 grams such that the total mass of the weights 56 and the apparatus 50 is 289 grams. A fluid insult of 15 g of 0.9 weight percent sodium chloride solution is then applied to the sample 60 through the cylinder of the apparatus and the time for the liquid to be transferred into the sample recorded. Divide the total charge of 15 g by the intake time to obtain the intake rate.

Repeat the intake test on each sample 3 times for a total insult of 45 g sodium chloride solution applied to the sample. Allow for about 15 minutes between insults. The calculations for intake rate are the same each time. When the tests are complete conduct a saturation test on the sample to determine total saturation capacity.

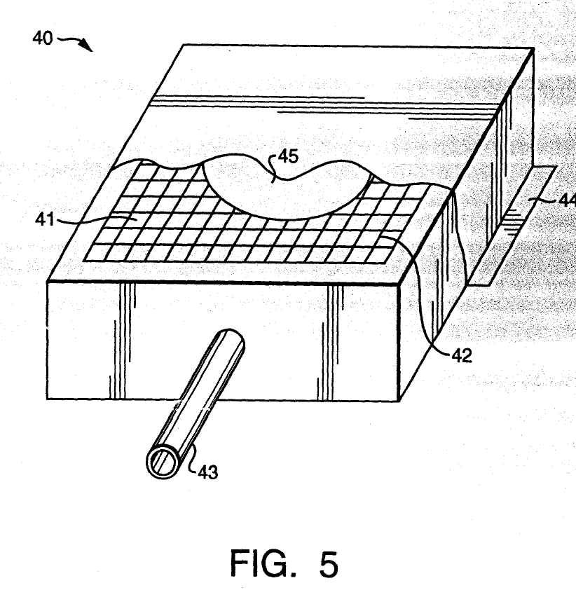

The liquid saturated retention capacity is determined as follows. The material to be tested is weighed and submerged in an excess quantity of 0.9 wt % NaCl solution at standard conditions; 22.2° C., 50 percent relative humidity, 760 mmHg pressure. The material to be tested is allowed to remain submerged for about 20 minutes. After the 20 minute submerging, the material 45 is removed and, referring to FIG. 5, placed on a vacuum apparatus 40 having an upper surface 41 with 6.35 mm (0.25 inch) diameter openings and covered with 100 mesh screen 42 which, in turn, is connected to a vacuum source 43 and covered with a flexible rubber dam material 44. A vacuum of about 698 mmHg (13.5 pounds per square inch) is drawn on the vacuum apparatus for a period of about 3 minutes. The material being tested is then removed from the apparatus and weighed. The amount of liquid retained by the material being tested is determined by subtracting the dry weight of the material from the wet weight of the material (after application of the vacuum), converting the weight to milliliters by using the density of the test liquid, and is reported as the liquid saturated retention capacity in milliliters of liquid retained. For relative comparisons, the weight of liquid held (wet weight after application of vacuum minus dry weight) can be divided by the weight of the material 31 to give specific liquid saturated retention capacity in grams of liquid retained per gram of tested material.

Because intake rate changes as a function of saturation, the data should be normalized to a common set of criteria. Using the saturated capacity of the composite, determine the percent saturation of sample upon each insult. For example, a 15 g insult and a 45 g saturation capacity yields 33 percent saturation. Plot the intake rate of an absorbent composite as a function of the percent saturation. The value of percent saturation used to plot the intake rate against is the average value between the saturation at the beginning and end of the insult. Interpolate the effective intake rate at the 50 percent composite saturation level. The final step is to generate a plot of the intake rate at 50 percent composite saturation versus the capacity of the absorbent composite.

### DETAILED DESCRIPTION OF THE INVENTION

The web of this invention includes superabsorbent fibers, binder and optionally additional fibers and particulate superabsorbent as may be needed to achieve the objectives of the designer. Additional fibers include synthetic fibers and natural fibers.

Synthetic fibers include those made from polyamides, polyesters, rayon, polyolefins, acrylics, Lyocel regenerated cellulose (Rayon) and any other suitable synthetic fibers known to those skilled in the art and are not superabsorbent. Synthetic fibers may also include kosmotropes for product degradation. These fibers may be used in the web to increase the bulk of the web in order to provide more void space within the web for liquid passage.

The fabric used in the practice of this invention may have hydrophilic natural fibers such as pulp and cotton. These fibers, as well as synthetic fibers like Rayon which are also hydrophilic, are useful in the practice of the invention in order to control the overall wettability of the web.

Preferred binder fibers for inclusion are those having a relatively low melting point such as polyolefin fibers. Lower melting point polymers provide the ability to bond the fabric together at fiber cross-over points upon the application of heat. In addition, heterogeneous fibers having a lower melting polymer, like conjugate and biconstituent fibers are suitable for practice of this invention. Fibers having a lower melting polymer are generally referred to as “fusible fibers”. By “lower melting polymers” what is meant are those having a melting temperature less than about 175° C. It should be noted that the texture of the absorbent web can be modified from soft to stiff through selection of the fusion and quenching behavior of the polymer.

Exemplary insoluble binder fibers include conjugate fibers of polyolefins, polyamides and polyesters. Three suitable binder fibers are sheath core conjugate fibers available from KoSa Inc. (Charlotte, N.C.) under the designation T-255 and T-256, both with a polyolefin sheath, or T-254, which has a low melt co-polyester (Co-PET) sheath. Many suitable insoluble binder fibers are known to those skilled in the art, and are available by many manufacturers such as the Chisso Corporation of Moriyama, Japan, and Fibervisions LLC of Wilmington, Del. A common bicomponent binder fiber is a side-by-side or sheath/core polyethylene/polypropylene fiber.

Superabsorbents fibers that are useful in the present inventions can be chosen from classes based on chemical structure. These include superabsorbents with low gel strength, high gel strength, surface cross-linked superabsorbents, uniformly cross-linked superabsorbents, or superabsorbents with varied cross-link density throughout the structure. Superabsorbents may be based on chemistries that include but are not limited to acrylic acid, iso-butylene/maleic anhydride, polyethylene oxide, carboxy-methyl cellulose, poly vinyl pyrrollidone, and poly vinyl alcohol. The superabsorbents may range in rate from slow to fast and may be in various length and diameter sizes and distributions. The superabsorbents may be in various degrees of neutralization. Neutralization occurs through use of counter ions such as Li, Na, K, Ca.

An exemplary superabsorbent fiber was obtained from Camelot Technologies of High River, Alberta, Canada, and is designated FIBERDRI® 1241. Additionally available is FIBERORI® 1161. Further examples of fibrous superabsorbents were obtained from Technical Absorbents, Ltd., of Grimsby, United Kingdom, and are designated OASIS® 101, OASIS® 102 and OASIS® 111. Additional fibrous superabsorbents not listed here can be useful in the present inventions.

Particulate superabsorbents may also be added to enhance the capacity of the absorbent composite. Examples of such superabsorbents include FAVOR® SXM 880 from Stockhausen in Greensboro, N.C. and DryTech® 2035 from Dow Chemical in Midland, Mich. Other types of particulate superabsorbents not listed here may also be useful in the present inventions. One process for producing such a fibrous structure is described in U.S. Pat. No. 6,024,813, issued Feb. 15, 2000 to Groeger et al. and assigned to AQF Technologies LLC of Charlotte, N.C., includes carding a web of staple fibers and distributing particulate matter therein. Another method that may be used is that taught in U.S. patent application Ser. No. 091209,044 filed Dec. 9, 1998 to Varona et al and assigned to Kimberly-Clark Worldwide, Inc.

Z-directional orientation of the fibers in the web is important in order to provide a high intake rate. Without wishing to be bound by theory, it is thought that liquid coming into contact with the ends of the fibers in the web is quickly wicked into the web and moved lower into the structure. Also, the low web densities that are typically achieved by the Z-directional orientation of fibers allow the superabsorbent in the interstitial space sufficient freedom to absorb liquid and swell effectively.

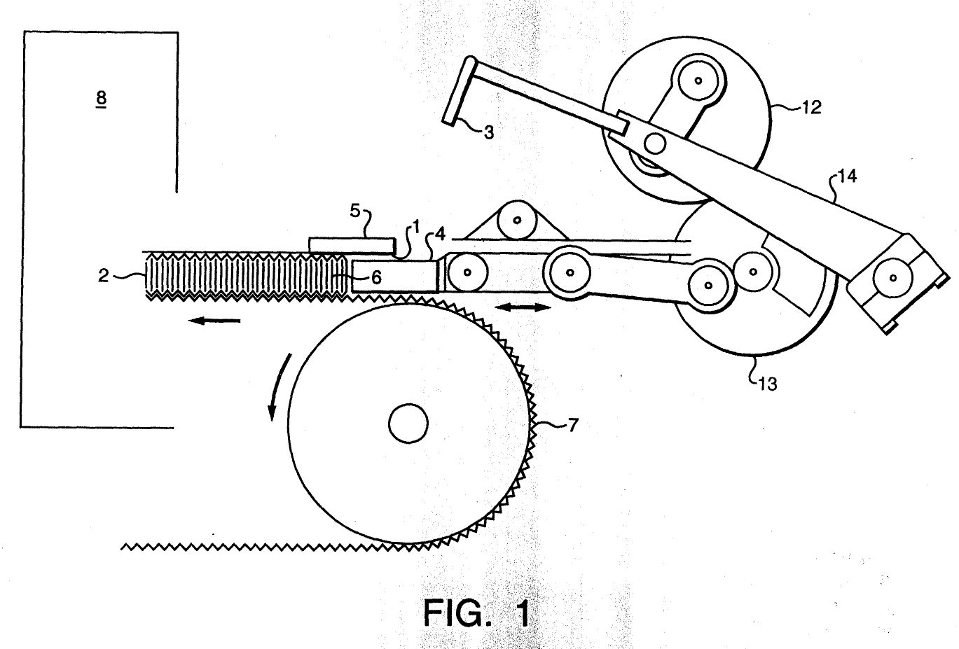

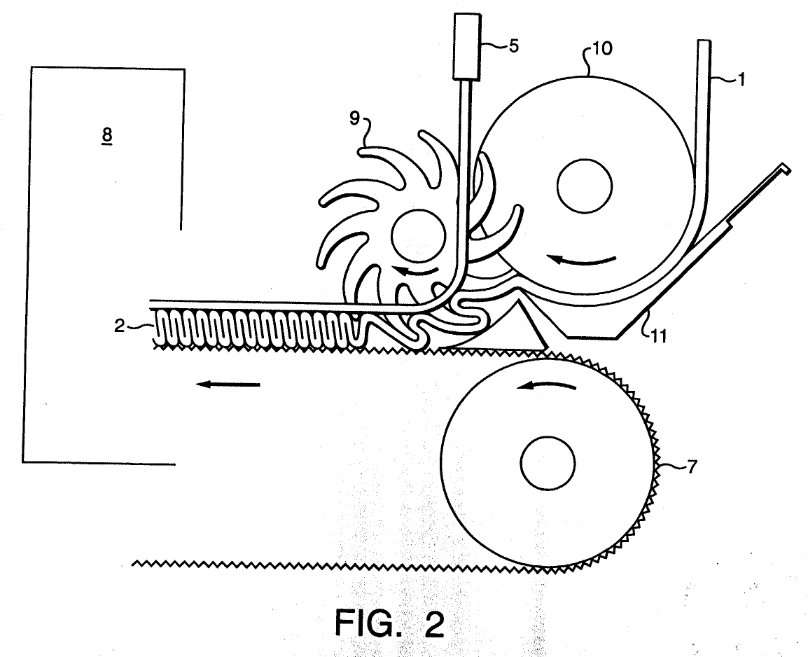

A number of methods are available to produce Z-directionally oriented webs. A particularly suitable method for producing Z-directionally oriented fiber webs may be found in the October 1997 issue of Nonwovens Industry magazine at page 74 in an article by Krema, Jirsak, Hanus and Saunders entitled “What's New in Highloft Production?” as well as in Czech patents 235494 entitled “Fibre Layer, Method of its Production and Equipment for Application of Fibre Layer Production Method” issued May 15, 1995 and 263075 entitled “Method for Voluminous Bonded Textiles Production” issued Apr. 14, 1989. The vibrating lapper (FIG. 1) and the rotary lapper (FIG. 2) therein described are commercially available from Georgia Textile Machinery of Dalton, Ga., USA, and STRUTO LLC of the Czech Republic.

In FIG. 1, the vibrating lapper has a reciprocating comb 3 attached to an arm 14 which is in turn driven by a first bell-crank mechanism 12. The gear driving the first bell crank mechanism 12 meshes with a gear driving a second bell-crank mechanism 13, which causes reciprocating movement of a presser bar 4 (which preferably incorporates a series of needles). As the web 1 is introduced onto the conveyor belt 7, the comb 3 and the presser-bar 4 are alternatingly driven by the bell-crank mechanisms, into and out of engagement with the web 1 so that the comb 3 produces pleats in the web 1, and so that the presser-bar 4 pushes the web 1 along a guide board 6 and compresses the pleats between the wire guide 5 and the conveyor belt 7. This results in a pleated web 2, which issues from the vibrating perpendicular lapper as shown. The conveyor belt 7 brings the pleated fiber web 2 into a bonding device 8, which typically functions either thermally or mechanically.

The rotary lapper shown in FIG. 2 feeds the carded web 1 between a feeding disc 10 and a feeding pan 11 and into the working disc teeth 9. The folds are created in the carded web 1 as it passes between the teeth 9 producing a perpendicularly laid fiber batt 2, which is transported between a conveyor belt 7 and a wire guide 5 towards a bonding device 8. The rotating lapper process and variants are further described in European patent application EP 0516964 B1 which teaches that fabrics so produced are useful primarily in the clothing industry as heat insulating lining materials, in the furniture industry as elastic fillers, in the automotive and construction industries as thermal and noise insulation, etc.

The use of perpendicularly laid fibers to form fabrics, according to the definition above, has been known for production of carpet under pads, sleeping bag insulation and sound insulation where basis weights were considerably higher than that permissible for personal care products which must be lightweight and comfortable. Z-directional fabrics have been investigated previously for personal care products wherein the fibers provide superior fluid movement. U.S. Pat. Nos. 4,578,070 and 4,681,577 for example, teach aligning the corrugations parallel to the longitudinal axis of a personal care product. U.S. Pat. No. 4,886,511 teaches the use of elasticized strands across the crotch of a diaper so as to corrugate the product. EP 0767649 A1 describes a pleated front covering layer for a sanitary napkin with longitudinal channels on the surface. U.S. Pat. No. 5,695,487 teaches the use of meltblown webs for such fabrics wherein the fibers were aligned in the longitudinal direction.

The webs which may be subjected to the Z-directional orientation process may be produced by a variety of processes including airlaying, bonded carded web processes, spunbonding, meltblowing and coform processes. The webs may be made from a variety of fibers and mixtures of fibers including superabsorbent fibers, synthetic fibers, natural fibers and binder fibers. The fibers in such a web may be made from the same or varying diameter fibers and may be of different shapes such as pentalobal, trilobal, elliptical, round, etc.

A number of samples were produced in order to test the properties of the webs of this invention. Samples 1-5 were made by an airforming method using a laboratory airforming handsheet unit. This airforming unit produced an intermingled fibers and superabsorbent particles directly onto a porous sheet of tissue. The web so produced was 3 inches (7.62 cm) in diameter and after formation another layer of the same tissue was placed on top of the web. The sample was then compressed to a density of 0.2 g/cc using a Carver Laboratory Press, Model 2518, made by Fred S. Carver Inc., Menomonee Falls, Wis. The tissue used was 9.8 pound (4.45 kg) White Forming tissue from American Tissue Inc. of Neenah, Wis.

Samples 1-3 contained particulate superabsorbent DryTech® 2035 from Dow Chemical Corp. of Midland, Mich. in an amount of 30, 50 and 60 weight percent respectively and the balance was Caressa 1300 pulp available from Buckeye Inc., of Memphis, Tenn.

Sample 4 contained FAVOR® SXM 880 particulate superabsorbent, available from Stockhausen Inc., of Greensboro, N.C. Sample 5 contained FAVOR® 9543 particulate superabsorbent from Stockhausen. Samples 4 and 5 contained 50 weight percent superabsorbent and 50 weight percent Caressa 1300 pulp.

Sample 6 was a 3 inch (7.6 cm) diameter circle of material, die cut from a point 6.5 inches (16.5 cm) from the front end of the absorbent pad, centered in the cross-direction. The material was cut from a commercially produced PAMPERS® Premium Step 2 diaper from the Proctor and Gamble Company of Cincinnati, Ohio. The diaper had a bag code of 1095U011162039. After the die cutting of the sample, all layers of the product were removed except for the superabsorbent and fluff layer.

Sample 7 was a 3 inch (7.6 cm) diameter circle of material, die cut from a point 6.5 inches (16.5 cm) from the front end of the absorbent pad, centered in the cross-direction. The material was cut from a commercially produced HUGGIES® Supreme Step 3 diaper from the Kimberly-Clark Corporation of Neenah, Wis. The diaper had a bag code of NM034102b 0545-1900. After the die cutting of the sample, all layers of the product were removed except for the superabsorbent and fluff layer.

Samples 8-10 were made into a Z-directionally oriented web using the vibrating lapper as described above with respect to FIG. 1 and contained OASIS® 102 superabsorbent fiber. Samples 8 and 9 contained 50 weight percent fibrous superabsorbent and sample 10 contained 70 percent fibrous superabsorbent. Sample 8 contained 50 weight percent 3 denier PP/PE bicomponent binder fiber from Chisso. Sample 10 contained 30 percent 3 denier PP/PE bicomponent binder fiber from Chisso and 20 percent Rayon fiber (merge 18453) from Courtalds Corporation of Grimsby, UK. Sample 10 contained 30 percent 3 denier PP/PE bicomponent binder fiber from Chisso.

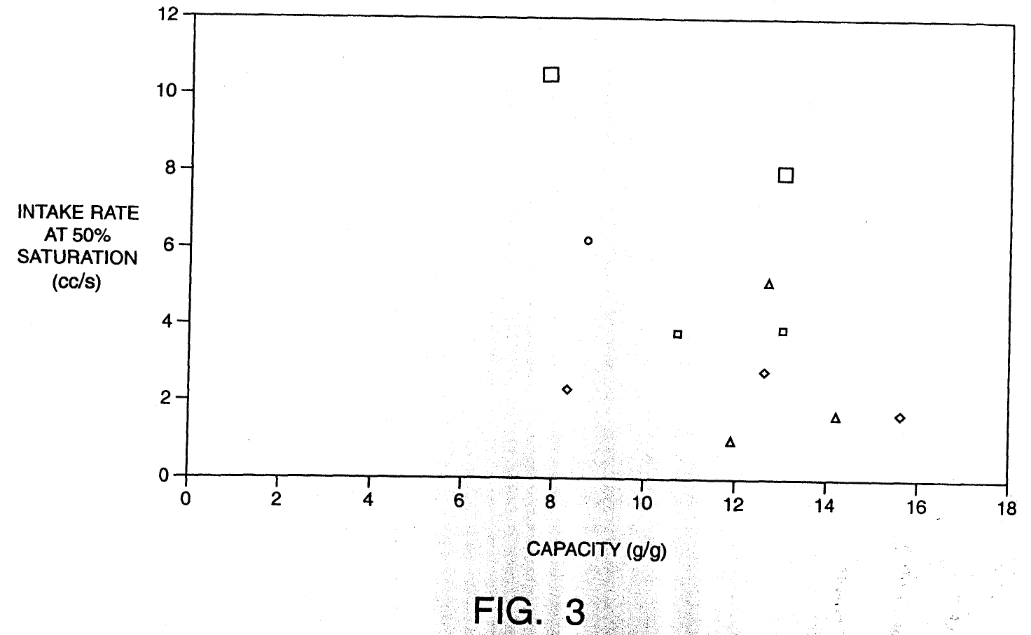

The above samples were tested according to the intake and capacity tests described above and the results given in Table 1. In Table 1 the capacity is given in g/g and the intake rate in cc/sec. The results are also given graphically in FIG. 3 where intake is on the Y-axis at 50 percent saturation in cc/s and capacity is given on the X-axis in g/g. On the graph of

FIG 3, the diamonds are samples 1-3, the open squares are samples 4-5, the triangles are the commercial diapers, the solid circle is sample 9, and the solid squares are the samples of this invention, 8 and 10.

TABLE 1

| Sample | Capacity | Intake Rate |
|---|---|---|
| 1 | 8.3 | 2.3 |
| 2 | 12.6 | 2.8 |
| 3 | 15.6 | 1.7 |
| 4 | 13 | 3.9 |
| 5 | 10.7 | 3.8 |
| 6 | 14.2 | 1.7 |
| 7 | 11.9 | 1 |
| 8 | 7.8 | 10.5 |
| 9 | 8.7 | 6.2 |
| 10 | 13 | 8 |

As can be seen by the results, the webs made according to the invention, having a Z-directional orientation and the proper composition, have both high intake rates and high liquid capacities. The intake rate is at least 7 cc/s at 50 percent saturation, more preferably above 8 cc/s and still more preferably above 10 cc/s. The capacity is at least 4 g/g, more preferably above 6 g/g and still more preferably above 10 g/g. The upper limit for this invention, in these properties, is believed to be 15 cc/s intake rate at 50 percent saturation and 20 g/g capacity. Recognized in the instant invention has been the contribution of perpendicularly laid fibers to fluid intake as well as the liquid capacity of the superabsorbent fibers. The Z-directional orientation of the fibers also results in good mechanical compression resilience. These properties make the webs of the inventions ideal for use in personal care products.

As will be appreciated by those skilled in the art, changes and variations to the invention are considered to be within the ability of those skilled in the art. Examples of such changes and variations are contained in the patents identified above, each of which is incorporated herein by reference in its entirety to the extent consistent with this specification. Such changes and variations are intended by the inventors to be within the scope of the invention.

What is claimed is:

1) A nonwoven material for personal care products comprising superabsorbent fibers in an amount of at least 40 weight percent and binder in an amount of between 10 and 60 weight percent, in a Z-directionally oriented web.

2) The nonwoven of claim 1 further wherein said binder is synthetic bicomponent fibers.

3) The nonwoven of claim 2 further comprising natural fibers in an amount of at most 40 weight percent.

4) The nonwoven of claim 3 further comprising synthetic polymeric fibers selected from the group consisting of polyolefins, polyamides, polyesters, polyethers, polyethylene terephalate and combinations thereof.

5) A diaper comprising the material of claim 3.

6) A training pant comprising the material of claim 3.

7) An incontinence product comprising the material of claim 3.

8) A bandage comprising the material of claim 3.

9) A sanitary napkin comprising the material of claim 3.

10) A nonwoven material for personal care products comprising superabsorbent fibers in an amount of at least 50 weight percent, polypropylene/polyethylene bicomponent fiber in amount of at least 20 weight percent, in a Z-directionally oriented web.

11) The nonwoven of claim 10 further comprising natural fibers wherein said natural fibers are hydrophilic.

12) The nonwoven of claim 11 wherein said natural fiber is selected from the group consisting of cotton, pulp and rayon.

13) A diaper comprising the material of claim 12.

14) A training pant comprising the material of claim 12.

15) An incontinence product comprising the material of claim 12.

16) A bandage comprising the material of claim 12.

17) A sanitary napkin comprising the material of claim 12.

18) A nonwoven material for personal care products comprising superabsorbent fibers in an amount of at least 60 weight percent, polypropylene/polyethylene bicomponent fiber in amount of about 30 weight percent, and rayon fibers, in a Z-directionally oriented web.

19) A nonwoven material for personal care products comprising a Z-directionally oriented web having an intake rate at 50 percent saturation of at least 7 cc/s and a capacity of at least 4 g/g.

20) The nonwoven material according to claim 19 having an intake rate at 50 percent saturation of at least 8 cc/s and a capacity of at least 6 g/g.

21) The nonwoven material according to claim 19 having an intake rate at 50 percent saturation of at least 8 cc/s and a capacity of at least 10 g/g.

22) A nonwoven material for personal care products comprising superabsorbent fibers in an amount of at least 50 weight percent, polypropylene/polyethylene bicomponent fiber in amount of at least 20 weight percent, in a Z-directionally oriented web, wherein said web has an intake rate at 50 percent saturation of at least 7 cc/s and a capacity of at least 4 g/g.
